(19) United States
(12) Patent Application Publication
(10) Pub. No.: US 2002/0028446 A1
(43) Pub. Date: Mar. 7, 2002

(54) SYSTEM AND METHOD FOR MOLECULE SELECTION USING EXTENDED TARGET SHAPE

(76) Inventor: STUART A. KAUFFMAN, SANTA FE, NM (US)

Correspondence Address: PENNIE & EDMONDS LLP, 1667 K STREET NW, SUITE 1000, WASHINGTON, DC, 20006

(21) Appl. No.: 09/433,081

(22) Filed: Nov. 3, 1999

Related U.S. Application Data
(60) Provisional application No. 60/106,791, filed on Nov. 3, 1998.

(51) Int. Cl. C12Q 1/00; C12Q 1/68; C07K 14/00; G01N 33/53

(52) U.S. Cl. 435/6; 435/4; 435/7.1; 530/350

(57) ABSTRACT

The present invention is directed to production of a molecule having a predetermined property. In accordance with one embodiment, a library of initial candidate molecules that are at least somewhat dissimilar to a chosen target molecule or “targetshape” is generated. Variants of the initial candidates are generated and screened to identify intermediate candidates from among those variants that are either more or less similar to the targetshape. The process may be iterated by generating variants of the intermediate candidates and screening these variants to identify molecules further more or less similar to the targetshape.

This application claims the benefit under 35 U.S.C. § 119(e) of provisional application No. 60/106,791, filed Nov. 3, 1998, which is hereby incorporated by reference in its entirety.

### 1. INTRODUCTION

The present invention includes a means to obtain one or more initial candidate molecules that are at least somewhat dissimilar to a chosen target molecule or “targetshape”, to produce initial variants of the initial candidate molecules, and to screen or select candidates from among those variants for molecules that are either more or less similar to the targetshape.

### 2. BACKGROUND OF THE INVENTION

The new field of applied molecular evolution, or molecular diversity, is rapidly becoming of central importance in the generation of useful molecules for drugs, vaccines, biosensors, catalysts, and so forth. Molecular diversity is based on generating very large libraries of candidate compounds, up to 10¹⁵ for quasirandom single stranded RNA or DNA sequences, 10¹³ for phage displayed polypeptides, and into the millions for libraries of small molecules. These libraries are then screened or subjected to selection in order to find useful candidate compounds.

Typical screening procedures, as specified, e.g., in U.S. Pat. No. 5,824,514 to S. Kauffman and Balivet, incorporated herein by reference in its entirety, are based on the use, for example, of a ligand as the screen, and then screening for a novel molecule able to bind the ligand. For example, the ligand might be the estrogen receptor and phage display libraries are searched for novel peptides or polypeptides able to bind the estrogen receptor. Any such peptide or polypeptide is a candidate drug which might mimic, modulate, agonize, or antagonize the action of estrogen. In an equivalent procedure, the SELEX procedure, an RNA molecule able to bind a target is selected. Typically, once an initial set of candidate compounds is located, the candidates are in one form or another, “amplified” or replicated and then subjected to successive binding and amplification cycles in order to winnow down to good binding candidates. The U.S. Pat. No. 5,824,514, Patent No. WO9424314 to S. Kauffinan and J. Rebek, S. Brenner et al. Proc. Natl. Acad. Sci. USA, 1992, 89:5381, are hereby incorporated in their entireties as non limiting examples of generating, characterizing, and screening molecular diversity libraries.

The term in the art for the early stages of the drug development process is called “lead discovery,” and a successful candidate is referred to as a “lead.”

Three gaps in the current technology are becoming increasingly important to close:

1) Consider screening a population of molecules for candidates that mimic the shape and/or structure of some target compound. Present screening or selection techniques primarily identify candidates that are very close in shape and/or function to the target compound, e.g., candidates that efficiently bind to receptors or antibodies of the target compound. Therefore, molecules that are “close” in shape and/or structure to a viable mimic for the target molecule, but not similar enough to bind efficiently a target receptor or target antibody remain undetected by current screening procedures.

2) A second, pressing problem in the drug discovery field is referred to herein as “The Multiple Target Problem”. Typical drug compounds must satisfy a number of criteria. For example, a compound may be sought that selectively binds a particular receptor in preference to one or more different receptors, crosses cell membranes and nuclear membranes, survives oral ingestion, does not cross the blood brain barrier, shows good renal clearance, and does not exhibit a variety of cross binding properties to other molecules or sites that would cause further side effects. The process of further developing or modifying a lead compound to meet such criteria is often defined as lead optimization. Lead optimization is financially and labor intensive. For example, if the total cost of development of a drug, including clinical trials, is on the order of 200 to 300 million dollars, then the typical cost of lead discovery might be on the order of 1 million dollars, while lead optimization may typically cost 20 to 40 million dollars. That is, lead optimization is a far more expensive and complex step in the drug development process than lead discovery.

Indeed, the very field of molecular diversity is making the discovery of good leads ever easier, hence commoditizing the discovery of drug leads.

Problems 1 and 2 above are related: Solving the multiple target problem, in general, requires finding a set of initial candidate molecules or leads able to meet a number of different criteria. One would expect to find initial candidates that were only slightly able to perform several or all of the tasks, then optimizing one or more candidates until optimum (perhaps compromise) candidates were obtained. Thus, the capacity to find candidates to fulfill multiple tasks simultaneously is typically going to require the ability to locate and optimize molecules which are initially quite poor at all or most of the tasks.

3) In order to solve the multiple target problem, it will be necessary to generate ever “improved” libraries of candidate molecules “spotted into” the proper region of molecular shape space. Thus, suppose one wished to find a molecule able to bind the estrogen receptor and also able to bind some other receptor, X. Initial candidates might be poor at both tasks, so poor that one could not obtain binding either to the X or the estrogen receptor. It is an object of the present invention to detect molecules that are modestly close to being able accomplish both tasks, i.e., bind both the estrogen and X receptors. Then, the initial screen will have identified a good region of shape space where candidates to solve both tasks are located. Then, further screening or selection would be enabled by the capacity to generate a new library of candidate molecules in the vicinity of this good region of shape space and select or screen for candidates with improved capacities to accomplish both tasks. A succession of such steps, generating and testing new libraries directly or in part computationally, would then constitute a lead optimization procedure with respect to these two tasks.

### 3. SUMMARY OF THE INVENTION

One aspect of the present invention is to provide a method for obtaining molecular diversity comprising the steps of:

obtaining an intermediate set of molecules that bind at least one molecule belonging to an origin set of molecules; and

obtaining a terminal set of molecules that bind at least one molecule belonging to the intermediate set of molecules.

In another embodiment, the method further comprises the steps of:

replacing the intermediate set of molecules with the terminal set of molecules;

obtaining a new terminal set of molecules that bind to at least one molecule in the intermediate set of molecules;

replacing the terminal set of molecules with the new terminal set of molecules; and

repeating the replacing the intermediate set of molecules step and the obtaining a new terminal set of molecules step.

In another embodiment, the method comprises the further steps of:

selecting a subset of the terminal set of molecules having a predetermined characteristic;

replacing the origin set of molecules with the intermediate set of molecules;

replacing the intermediate set of molecules with the subset of the terminal set of molecules; and

obtaining a new terminal set of molecules that bind to at least one molecule in the intermediate set of molecules;

replacing the terminal set of molecules with the new terminal set of molecules; and

repeating the replacing the origin set of molecules step, the replacing the intermediate set of molecules step and the obtaining a new terminal set of molecules step.

In one embodiment of the present invention, the predetermined characteristic is the ability of a member of the terminal set of molecules to bind to at least one member of the intermediate set of molecules in substantially the same way as the member of the intermediate set of molecules binds at least one member of the origin set of molecules.

It is another aspect of the current invention to provide a method for drug lead discovery comprising the steps of:

obtaining an intermediate set of molecules that bind at least one molecule belonging to an origin set of molecules;

obtaining a terminal set of molecules that bind at least one molecule belonging to the intermediate set of molecules;

producing one or more first variants of the terminal set of molecules that are similar to members of the origin set of molecules; and

selecting one or more of the first variants having at least one desired characteristic.

It is another aspect of the present invention to provide a method for generating a molecule having a desired characteristic comprising the steps of:

obtaining a set of molecules that bind at least one molecule belonging to an origin set of molecules;

repeating said obtaining step from said set of molecules to generate a sequence of sets of molecules wherein the molecules in each of said sets bind to the molecules in a preceding one of the sets in the sequence;

combining one of said sets of molecules with a first group of molecules to create a plurality of product molecules;

selecting another of said sets of molecules in said sequence;

identifying one or more of said product molecules that bind at least one molecule in said selected set of molecules; and

repeating said combining step with said identified product molecules, said selecting step and said identifying step to generate the molecule with the desired characteristic.

Another aspect of the present invention is to provide a method for treating an animal having an autoimmune disease comprising the steps of:

isolating an antibody from said animal to form an origin set;

obtaining a first intermediate set of molecules that bind said origin set;

obtaining a first terminal set of molecules that bind said first intermediate set;

obtaining a second intermediate set of molecules that bind said first terminal set;

selecting therapeutic molecules in said first intermediate set and molecules in said second intermediate set; and

administering said therapeutic molecules to said animal.

It is another aspect of the present invention to provide a method for producing an autocatalytic system capable of producing a molecule having a desired property comprising:

selecting an origin set of molecules;

generating an intermediate set of molecules and a terminal set of molecules using the method for obtaining molecular diversity of the present invention;

combining said intermediate set and said terminal set of molecules to form a system;

providing matter and/or energy to said system; and screening said system for molecules having said desired property.

It is another aspect of the present invention a method of characterizing a first molecule comprising the steps of:

selecting a plurality of origin set molecules;

creating an intermediate set of molecules and a terminal set of molecules from each of said origin set molecules using the method for obtaining molecular diversity of the present invention;

identifying terminal sets of molecules having at least one molecule that binds said first molecule; and

identifying intermediate sets of molecules having at least one molecule that binds said first molecule.

### 4. BRIEF DESCRIPTION OF THE FIGURES

FIG. 1 is a flowchart representing an example of a process for obtaining a targetshape group.

FIG. 2 is a flowchart representing an example of a process for generating a series of directed chemical reactions.

FIG. 3 is a flowchart representing an example of a process for characterizing the relative shape or functionality of a molecule.

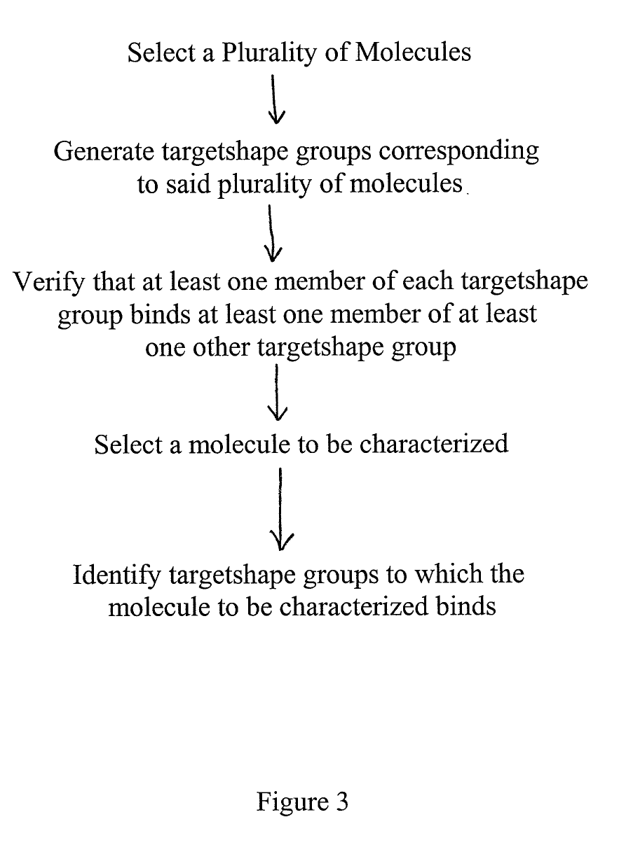

FIG. 4 is a flowchart representing an example of a process for treating autoimmune disease.

FIG. 5 is a flowchart representing an example of a process for generating an autocatalytic system capable of producing a molecule of interest.

### 5. DETAILED DESCRIPTION OF THE PREFERRED EMBODIMENT

The present invention has as its object, a means to obtain one or more initial candidate molecules, e.g., lead molecules in a drug discovery process, that are at least somewhat dissimilar to a chosen target molecule or “targetshape”, to produce initial variants of the initial candidate molecules, and to screen or select candidates from among those variants for molecules that are either more or less similar to the targetshape. The process of producing variants and screening or selecting from among the variants for molecules may by repeated at least once. Hence, the present invention provides a means to carry out an adaptive walk in molecular shape space to climb towards or away from close mimics of a given targetshape.

A further object of the present invention is to provide a means of generating diversity libraries of candidate molecules that are “focused” into a selected region of shape space. For example, without limitation, consider the problem of finding a molecule able to bind the estrogen receptor and also able to bind some other receptor, X. Initial candidates might bind so weakly to both receptors as to be undetectable. The present invention provides a means of identifying initial candidate molecules that are only modestly “close” to being able to bind both the estrogen and X receptors. These initial candidates likely occupy the same region of molecular shape space that is or would be occupied by improved candidates that are better able to bind both receptors. Improved candidates can be sought by generating a new library of candidate molecules focused in the same general vicinity of shape space as the initial candidates and selecting or screening for improved candidates better able to bind simultaneously both receptors. An iterative succession of such steps, obtaining and testing new libraries directly or in part computationally, constitutes a lead optimization procedure with respect to these two tasks.

A variety of characteristics may be used to select molecules according to the invention. According to one mode of carrying out the process according to the invention, the property serving as the criterion of selection is that of having at least one epitope similar to one of the epitopes of a given antigen or other molecule. According to another mode of the invention the criterion for selection may be the capacity of a molecule to bind a given antigen, other molecule, or surface. According to yet another mode, the criterion for selection may be the capacity of a molecule to displace a member of two or more bound molecules.

The property serving as the criterion for selection can be the capacity of the molecule to catalyze a given chemical reaction. For instance, for the production of several peptides and/or polypeptides, the said property can be the capacity to catalyze a sequence of reactions leading from an initial group of chemical compounds to at least one target compound.

The said property can also be the capacity to modify selectively the biological or chemical properties of a given compound, for example, the capacity to selectively modify the catalytic activity of a polypeptide or other molecular catalyst.

The said property can also be the capacity to stimulate, inhibit, or otherwise modify at least one biological function of at least one biologically active compound, chosen, for example, among the hormones, neurotransmitters, adhesion factors, growth factors, and specific regulators of DNA replication and/or transcription and/or translation of RNA.

The invention also has as its object the use of the molecule obtained by the processes of the invention, for the measurement, e.g., qualitatively, quantitatively, or both of an analyte or other target molecule.

According to a particularly advantageous mode of carrying out the invention, the desired characteristic of the molecule is the capacity to simulate or modify the effects of a biologically active molecule, for example, a protein, and screening and/or selection for clones of transformed host cells producing at least one peptide or polypeptide having this property, is carried out by preparing antibodies against the active molecule, then utilizing these antibodies after their purification, to identify the clones containing this peptide or polypeptide, then by cultivating the clones thus identified, separating and purifying the peptide or polypeptide produced by these clones, and finally by submitting the peptide or polypeptide to an in vitro assay to verify that it has the capacity to simulate or modify the effects of the said molecule.

The invention carries over to obtaining polypeptides by the process specified above and utilizable as chemotherapeutically active substances.

5.1 Molecular Diversity Libraries

5.1.1 Recombinant Techniques

A variety of means are available for the generation of molecular diversity libraries.

For example, and not by way of limitation, a process for obtaining DNA, RNA, peptides, polypeptides, or proteins through the use of transformed host cells containing genes capable of expressing these RNA's peptides, polypeptides, or proteins, i.e., by recombinant DNA techniques as described in U.S. Pat. No. 5,824,514 to S. Kauffman et al., U.S. Pat. No. 5,763,192 to S. Kauffman et al., U.S. Pat. No. 5,723,323 to S. Kauffman et al., M. Pavia et al. Bioorg. & Med. Chem. Ltrs., 1993, 3:387, and J. Devlin, et al. Science, 1990, 249:404 which are hereby incorporated by reference in their entireties. Using such techniques, a library of expression vectors containing stochastically generated polynucleotide sequences is formed. Host cells containing the vectors are cultured so as to produce peptides, polypeptides, or proteins encoded by the stochastically generated polynucleotide sequences. Screening or selection is carried out on such host cells to identify a peptide, polypeptide or protein produced by the host cells which has a predetermined property. The stochastically generated polynucleotide sequence which encodes the identified peptide, polypeptide, or protein is then isolated and used to produce the peptide, polypeptide, or protein have the predetermined property.

5.1.2 Random Chemistry

Another approach to generating a diversity of compounds is described in Patent No. WO9424314 to Kauffman and Rebek, incorporated herein by reference in its entirety, discloses the generation of new compounds using random chemistry, with or without enzymes, and the subsequent characterization or identification of compounds with a desired property.

In one random chemistry approach, a starting group of different organic molecules is provided. At least one chemical reaction is caused to take place with at least some of the different organic molecules in the starting group to create an intermediate reaction mixture having one or more organic molecules different from the organic molecules in the starting group. The step of causing at least one chemical reaction to take place is repeated at least once. Subsequent repetitions uses the reaction mixture of the previous step, and in the end produces a final reaction mixture as a result of the last repetition. The final reaction mixture is screened for the presence of the organic molecule having a desired property.

In another approach to random chemistry, a diversity of compounds is generated from a group of substrates which are subjected to a group of enzymes representing a diversity of catalytic activities. As used herein, the term “enzyme” includes enzymes (e.g., naturally or non-naturally occurring or produced), catalysts (e.g., catalytic surfaces), candidate catalysts and candidate enzymes (e.g., antibodies, RNA, DNA or random peptides/polypeptides). The substrates may have different or similar core structures, and similar or different functional groups as substituents. Alternatively, the substrates may have different or similar core structures and different or similar functional groups as substituents. The substrates may have similar or identical core structures, but a variety of different functional groups as substituents permitting the creation of a diversity of compounds centered around a particular compound or a particular class of compounds.

For example, one may react a group of different enzymes representing a diversity of catalytic activities under suitable conditions with a group of different substrates, thereby producing one or more organic molecules different from the enzymes and substrates in the reaction mixture; screen the reaction mixture for the presence of an organic molecule having a desired property; and isolate from the reaction mixture the organic molecule having the desired property. In another approach, one may react a group of different enzymes representing a diversity of catalytic activities under suitable conditions with a group of different substrates, thereby producing one or more organic molecules different from enzymes and substrates in the reaction mixture; screen the reaction mixture for the presence of an organic molecule having a desired property; and determine the structure or functional properties characterizing the organic molecule have the desired property.

Using a random chemistry approach, at least two ways are provided for generating a diversity of molecules, one which does not use enzymes, but uses a variety of possible adducts or other molecules which may undergo reactions with the initial molecule of interest, and also uses a variety of chemical reagents and physical conditions to drive the synthesis of a library of derivatized products of the initial molecule. Alternatively, the core initial molecule plus a set of candidate adducts and other molecules which may react with the initial molecule are used, but also included is a set of enzymes which may increase the rate of formation of the local high diversity library of derivatized forms of the initial compound. It will be readily appreciated by those of ordinary skill in the art that the methods for producing general high diversity libraries of product molecules and for producing local high diversity libraries of derivatized forms of an initial compound may be combined. For example, a new initial compound may be generated by the general procedure (e.g., substrates with different core structures). Such a new compound is then used, with or without derivatives, to generate a local high diversity library of derivatized forms of the compound. Further, it will be evident to those of ordinary skill in the art that libraries may be generated using a combination of random chemistry methods without enzymes and with enzymes.

5.1.3 Production of Antibodies

Described herein are methods for the production of antibodies capable of specifically recognizing one or more target epitopes or molecules. Such antibodies may include, but are not limited to polyclonal antibodies, monoclonal antibodies (mAbs), humanized or chimeric antibodies, single chain antibodies, Fab fragments, F(ab′)₂ fragments, fragments produced by a Fab expression library, anti-idiotypic (anti-Id) antibodies, and epitope-binding fragments of any of the above. Such antibodies are useful as shape complements to one or more target molecules as part of a molecular diversity library according to the invention.

For the production of antibodies to target epitope or molecule, various host animals may be immunized by injection with the target molecule a portion thereof. Such host animals may include but are not limited to rabbits, mice, and rats, to name but a few. Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (bacille Calmette-Guerin) and Corynebacterium parvum.

Polyclonal antibodies are heterogeneous populations of antibody molecules derived from the sera of animals immunized with an antigen, such as target molecule, or an antigenic functional derivative thereof. For the production of polyclonal antibodies, host animals such as those described above, may be immunized by injection with the target molecule supplemented with adjuvants as also described above.

Monoclonal antibodies, which are homogeneous populations of antibodies to a particular antigen, may be obtained by any technique which provides for the production of antibody molecules by continuous cell lines in culture. These include, but are not limited to the hybridoma technique of Kohler and Milstein, (1975, Nature 256:495-497; and U.S. Pat. No. 4,376,110), the human B-cell hybridoma technique (Kosbor et al., 1983, Immunology Today 4:72; Cole et al., 1983, Proc. Natl. Acad. Sci. USA 80:2026-2030), and the EBV-hybridoma technique (Cole et al., 1985, Monoclonal Antibodies And Cancer Therapy, Alan R. Liss, Inc., pp. 77-96). Such antibodies may be of any immunoglobulin class including IgG, IgM, IgE, IgA, IgD and any subclass thereof. The hybridoma producing the mAb of this invention may be cultivated in vitro or in vivo. Production of high titers of mAbs in vivo makes this the presently preferred method of production.

In addition, techniques developed for the production of “chimeric antibodies” (Morrison et al., 1984, Proc. Natl. Acad. Sci., 81:6851-6855; Neuberger et al., 1984, Nature, 312:604-608; Takeda et al., 1985, Nature, 314:452-454) by splicing the genes from a mouse antibody molecule of appropriate antigen specificity together with genes from a human antibody molecule of appropriate biological activity can be used. A chimeric antibody is a molecule in which different portions are derived from different animal species, such as those having a variable region derived from a murine mAb and a human immunoglobulin constant region.

Alternatively, techniques described for the production of single chain antibodies (U.S. Pat. No. 4,946,778; Bird, 1988, Science 242:423-426; Huston et al., 1988, Proc. Natl. Acad. Sci. USA 85:5879-5883; and Ward et al., 1989, Nature 334:544-546) can be adapted to produce antibodies to one or more target molecules. Single chain antibodies are formed by linking the heavy and light chain fragments of the Fv region via an amino acid bridge, resulting in a single chain polypeptide.

Antibody fragments which recognize specific epitopes may be generated by known techniques. For example, such fragments include but are not limited to: the F(ab′)₂ fragments which can be produced by pepsin digestion of the antibody molecule and the Fab fragments which can be generated by reducing the disulfide bridges of the F(ab′)₂ fragments. Alternatively, Fab expression libraries may be constructed (Huse et al., 1989, Science, 246:1275-1281) to allow rapid and easy identification of monoclonal Fab fragments with the desired specificity.

5.2 Detection of Molecules and Molecular Binding

A variety of means are available which allow characterization, e.g., measurement quantitatively, qualitatively, or both, of low concentrations of one or more species of a desired molecule in a mixture of molecules generated by the methods provided herein. A variety of means are also available which allow characterization of binding or affinity between molecules.

In general, the methods of the invention comprise ascertaining the presence of a molecule having a desired property and/or measuring the abundance of a molecule having a desired property in a set or mixture of molecules generated by the methods provided herein.

A variety of cell systems are well known to those of ordinary skill in the art which allow measurement of low concentrations of ligands, e.g., ligands binding a hormone receptor. In this regard, for example, a system has been developed which clones human G peptide hormone receptors into frog melanocytes (Lerner, Proc. Natl. Acad. Sci. USA).

The hormone receptors, typically located in the cell membrane, respond to binding of the corresponding hormone, but trigger a cell response releasing or reabsorbing melanophores. In a forty minute reversible cycle, cells darken dramatically, then can be induced to lighten in color again. Response of the cell depends upon the affinity of the hormone for the receptor. Typical responses occur in the nanomolar to 100 picomolar hormone concentration range. For some hormone receptorhormone pairs where affinity is higher, response occurs in the picomolar hormone concentration range. This cell system is an example of an assay system which allows measurement, in a mixture of molecules, of one or more species of ligands able to bind to the receptor. The set of molecule ligands able to bind the receptor are then the ligands of interest, for they are candidates to act as drugs by antagonizing, agonizing, substituting for, or modifying the effects of the natural hormone. Alternatively, according to the methods of the invention, the ligands of interest may be those not binding the receptor.

A second example of a cell assay is that available commercially from Molecular Devices (Palo Alto, Calif.). It consists of an array of chemfets which respond to very small changes in local pH. In turn, these small pH changes reflect the altered metabolic activity of a population of cells upon receipt of some molecular signal, such as a hormone binding its receptor. For example, cell assays in which a hormone binds a receptor are known to those of ordinary skill in the art and allow nanomolar or subnanomolar concentrations of the hormone ligand to be measured. A preferred means of using the present invention consists in exposing such cells to a high diversity library of molecules or target shape set of molecules generated by the methods provided hereinl to ascertain the presence of or measure the abundance of one or more species of molecules able to trigger the cell response. That set of molecules, each of which is highly likely to bind the hormone receptor are the molecules of interest.

Another example is to use blast B cells, which on their surface express antibodies directed to a molecule of interest, to detect in a high diversity library the presence of molecules which sufficiently mimic the molecule of interest to be able to bind to its antibody on a B cell. Thus, an animal is immunized with a molecule of interest and the early B cells isolated. A high diversity library of molecules generated by the methods provided herein is screened using the population of B cells. For example, binding may stimulate cell cycling or division by the last B cell bound. Cell cycling or division may be detected by means known in the art.

Alternatively, a variety of assays to detect the presence of a ligand of interest exist which are based on direct binding assays. Thus, for example, a receptor for a hormone can be used directly to detect binding of a radioactivity labeled ligand. Other means, known in the art, to accomplish this include the following:

(i) The estrogen receptor is used as a non-limiting example. The cloned receptor can be affixed to a flat surface, for example, a filter. Very high specific activity estrogen is prepared, and bound to the receptor population. This set of bound receptors is then used in a competitive assay. The bound receptors are exposed to a library of compounds generated by the methods of the present invention. If the library contains ligands which also bind the estrogen receptor, those ligands will compete with the radioactively labeled estrogen itself for the receptors. Hence the radioactively labeled estrogen will be competitively displaced from the receptors and can readily be detected by means known in the art. Thus, this assay allows detection of one or more species of ligands in the mixture which compete with estrogen for the estrogen receptor. This set of ligands is the set of interest, as they are candidates to be drugs mimicking or antagonizing estrogen.

(ii) The estrogen receptor is again used as a nonlimiting example. By means known in the art, one raises antibody molecules which are able to bind the receptor when the receptor is not bound by estrogen, but not bind the receptor when occupied by estrogen. Alternatively, one generates antibody molecules which bind the estrogen receptor only when the receptor itself does bind estrogen. These antibody molecules can then be decorated with reporter groups by a variety of means known in the art, and used to detect the presence of one or more ligand species in a library of high diversity, which bind to the estrogen receptor. In the case of antibodies which only bind the receptor if the receptor is itself unbound by estrogens, one tests for loss of antibody binding in the presence of the library of compounds and in the simultaneous absence of estrogen. In the case of antibodies which bind the receptor only if the receptor is bound by estrogen, one tests for an increase in binding of the antibody in the presence of the receptor and high diversity library.

(iii) In order to detect ligands in a high diversity library which are candidates to mimic or antagonize the action of a given hormone or other molecule of interest, it is advantageous to generate one or more monoclonal antibodies which bind the hormone or other molecule of interest. This set of monoclonal antibodies can then be used, rather than a receptor, for the target molecule that is to be mimicked, in binding assays such as those noted above to detect the presence of one or more ligand species in the reaction mixture which are candidates to mimic or antagonize the action of the target molecule. An advantage of this procedure is that a receptor for the target molecule need not be available. Use of a set of monoclonal antibodies is advantageous because, a priori, it is not certain which molecular feature, or epitope, of the target molecule mediates its biological action. Use of a set of monoclonal antibodies, each responding to a different epitope on the target molecule, enhances the probability that the ligands detected in the high diversity library will include those which mimic the biologically important epitope of the target. In some cases it may be possible to selectively use only those monoclonal antibody molecules which bind to the known important epitope of the target molecule.

(iv) Means are established in the art to measure protein-protein binding based on plasmon resonance and detection of a shift in refractive index. In a detection system developed by Pharmacia (Piscataway, N.J.), a monoclonal antibody, or a hormone receptor is layered onto a gold chip. Binding of hormone, or other ligands to a receptor, is measured in very low concentrations (e.g., in the nanogram range or less). Thus, any receptor, or antibody, or other “shape complement” of a target molecule of interest can be placed on the gold chip, the latter can be exposed to a high diversity library, and the presence of binding species can be measured quantitatively, qualitatively, or both.

Another example of direct measurement of ligand-binding, which the applicant believe was developed by Evotech, can measure ligand binding in the femtomolar range.

A variety of approaches for characterizing molecular binding are based on fluorescence correlation spectroscopy. For example, Rudolph Rigler 1995, J. Biotechnology, 41:177 has reviewed fluorescence correlation approaches to measuring molecules and binding of molecules. In one approach, a laser beam is focused to a radius of less than about 1 micron. Fluorescent molecules or molecules labeled with fluorescent tags can be measured at femtomolar concentrations, (10⁻¹⁵ M), in tens of seconds. Binding of a fluorescent molecule or a molecule labeled with a fluorophore to another molecule can be characterized, e.g., measured qualitatively, quantitatively or both because of the reduced diffusion coefficient of the bound molecules compared to the unbound molecules. Similar approaches based on the different electrophoretic mobilities of bound and unbound molecules are known in the art. Competitive assays in which a molecule displaces a member of at least two bound molecules can be used to assess the relative binding efficiency or affinity of a set of molecules for one or more other molecules.

Thus, for example, if estrogen is a target molecule, and a small RNA aptomer is a shape complement which binds estrogen, then fluorescently labeled versions of that RNA aptomer can be used in a fluorescence correlation approach. An estrogen-mimic which binds the fluorescently labeled RNA will slow its diffusion as detected in the laser system. Thus estrogen-mimics at very low, 10⁻¹⁵ M or femtomolar, concentrations can be detected. Alternatively, one may begin with a number of complexes comprising estrogen and the fluorescently labeled RNA aptomer. Adding one or more molecules that compete with the RNA aptomer for binding sites on estrogen can be detected by the appearance of unbound labeled RNA aptomer. One or ordinary skill in the art recognizes that a number of possible approaches for detecting the binding and binding characteristics using a fluorescence based approaches are possible.

A further means to detect ligands of interest at very low concentrations consists in seeking ligands which block a DNA polymerase. By blocking the DNA polymerase chain reaction (PCR) enzyme, amplification of the DNA can be blocked. Since PCR amplification can yield billions or more copies of the initial DNA sequence, blocking PCR amplification yields a readily detectable signal of a ligand which blocks the polymerase. Clearly, this method generalizes to other means to amplify DNA, RNA, or DNA- or RNA-like molecules such as ligation amplification, and extends to general means to block polymerases directly or indirectly with ligands of interest.

As described herein, compounds of interest may act as catalysts for a desired reaction, or as cofactors with other molecules to form an active catalyst. Other molecules may act as inhibitors of enzymes. In order to exclude the possibility that the enzymes or catalysts are found among the candidate set of enzymes which may have been used to generate the compounds of interest, the latter set of enzymes can be quantitatively removed from the high diversity library by, for example, affinity columns bearing molecules directed to a constant part of each of the set of enzymes, or other means known in the art. The resulting high diversity library itself is then assayed for candidates of interest.

Detection of molecules able to inhibit an enzyme may proceed by detecting ligands able to bind the enzyme, as described above. Identifying molecules which are candidates to catalyze a reaction alone or as a cofactor may proceed by testing high diversity libraries of the invention alone, or in the presence of a helper molecule, say a protein, for which a desired molecule will be a cofactor. The system is tested for the presence of ligands able to bind a stable analogue of the transition state of the reaction. Such binding molecules are the candidate catalysts or cofactors sought, for they are candidates to catalyze the reaction itself.

Alternatively, a variety of means are known in the art which allow detection of the products of a catalyzed reaction itself. For example, chromogenic or fluorogenic substrates for a variety of reactions of interest are available. Catalysis of the reaction increases the rate of formation of the colored or fluorescent product. Alternatively, assay systems are available or readily prepared which detect the presence of a product molecule because that product molecule binds a receptor an antibody molecule, or other shape complement. Thus, detection of higher rates of formation of that product molecule demonstrates that the reaction itself was catalyzed.

5.3 Characterization of Molecular Libraries

Following the generation of high diversity libraries of compounds and the screening for the presence of compounds having properties of interest, compounds of interest may be characterized with or without isolation. A variety of means, including those known in the art, are available to characterize or isolate such compounds of interest.

Characterization and/or isolation depend upon the information desired and can be carried out at different mole abundances of the target molecule of interest. For example, using modem mass spectrographic analysis, about 10⁻¹⁵ to 10⁻¹⁸ moles can be assayed for mass and charge, then fragmented in a variety of ways known in the art and the fragments assayed for mass and charge. Using such data, it is possible to derive the structure of the molecule of interest. For example, ligands of interest may be isolated by binding to a given hormone receptor, or monoclonal antibody, then the liganding molecules released by means known in the art and finally characterized analytically. One means comprises attaching a target receptor or antibody to a solid support. A reaction mixture or subset thereof is contacted with the solid support. Those molecules that are bound will be retained, while the non-bound molecules are readily separated from the solid support. The molecules of unknown structure which have been retained, are then eluted. The freed molecules are characterized analytically, e.g., by mass spectroscopy, NMR, IR, UV, and may be synthesized in batch quantities.

Kibbey et al. U.S. Pat. No. 5,670,054, disclose an automated method of sample identification, purification and quantitation wherein a first HPLC column with defined operating parameters is used to separate a small portion of an impure mixture into its constituent components; the individual components corresponding to the eluting zones of the separated mixture are characterized by mass spectrometry; the chromatographic and mass spectroscopic data generated are stored in digital format, for example one compatible with commercial chromatography software, and the data is used to guide the purification of the remaining sample; the remaining sample is injected on a semi-preparative, or preparative HPLC column; an analog detector output of the semi-preparative, or preparative HPLC system is digitized and evaluated electronically with the previously generated chromatographic and mass spectroscopic data; when elution of a sample component peak corresponding to a desired product peak is sensed, a mechanically actuated, liquid switching value (i.e., a pneumatic or electronic switching valve) is actuated to divert the column eluate from waste to a fraction collection device; and when the end of product peak elution is sensed, the switching valve is actuated to divert the column eluate back to waste collection. The system enables rapid purification of samples in quantities useful for screening of diversity libraries while involving minimal operator input and minimum fraction collection equipment.

In other cases, the concentrations of molecules of interest in the high diversity library will allow detection of their presence, but may be too low for further isolation or characterization. A preferred procedure called “sib selection” allows ready winnowing of the set of candidate enzymes, the set of founder substrates, and the set of reaction conditions and chemical reagents to smaller sets. This winnowing simultaneously reduces the side products generated in the high diversity library, increases the concentration of the target molecule of interest, and identifies the subset of candidate enzymes which catalyze the pathway leading to synthesis of the target molecule, and identifies the set of founder substrates required for synthesis of the desired target. Thus, this sib selection procedure is a means to generate a previously unknown molecule of interest, as well as identify both that molecule and the substrates and enzymes needed to form that molecule.

5.4 Targetshape Groups

A targetshape of molecules, e.g., molecular diversity library, according to the present invention comprises a group of n sets of molecules si (i=0, . . . , n), wherein each set si contains at least 1 molecule. Set s0 generally contains one compound and represents the center or “targetshape” of the group of n sets of compounds. The group of n sets of molecules corresponding to set s0 and having set s0 at its center or origin is referred to as targetshape s. Each set of molecules may also be referred to as a “ring” or “shell.” A given ring si is said to have a higher order than ring s(i−1).

Initially, to obtain or generate members of targetshape set s1, a molecular diversity library composed of DNA, RNA, peptides, polypeptides, small molecules, or other compounds is generated or obtained and screened to obtain a set of molecules s1 able to bind a predetermined targetshape or set s0. Alternatively, or in addition, members of s1 may be found by using members of targetshape set s0 to raise antibodies against s0. Given that the members of set s1 bind members of s0, members of s1 generally have at least one epitope or shape feature that is at least somewhat complementary to at least one epitope or shape feature of members of s0.

Members of targetshape set s2 may be found by generating or obtaining and screening a molecular diversity library for molecules able to bind s2 and/or members of s2 may be found by using members of targetshape s1 to raise antibodies against s1.

Typically, for targetshape sets of order i≧2, any given set si includes a subset of members, si′, each of which bind at least one member of set s(i−1) byway of substantially the same epitope or molecular shape feature as those members of set s(i−1) bind at least one member of set s(i−2). Competitive binding assays may be used to identify members of each subset. For example, members of subset s2′ may be discriminated from the remainder of set s2 because s2′ and s0 will compete for the same binding site on one or members of s1. Given that members of s2′ and s0 compete for the same binding site on at least one member of s1, members of s2′ generally have at least one molecular epitope or shape feature that is similar to at least one molecular epitope or shape feature of s0. Thus, members of s2′ substantially correspond to mimics of s0.

Selecting members of si′ by competitive displacement of members of si off s(i−1)′ using members of s(i−2)′ is analogous to the concept of internal images in the immune system in second rank antiidiotypes and generally corresponds to the search for shape mimics using molecular diversity.

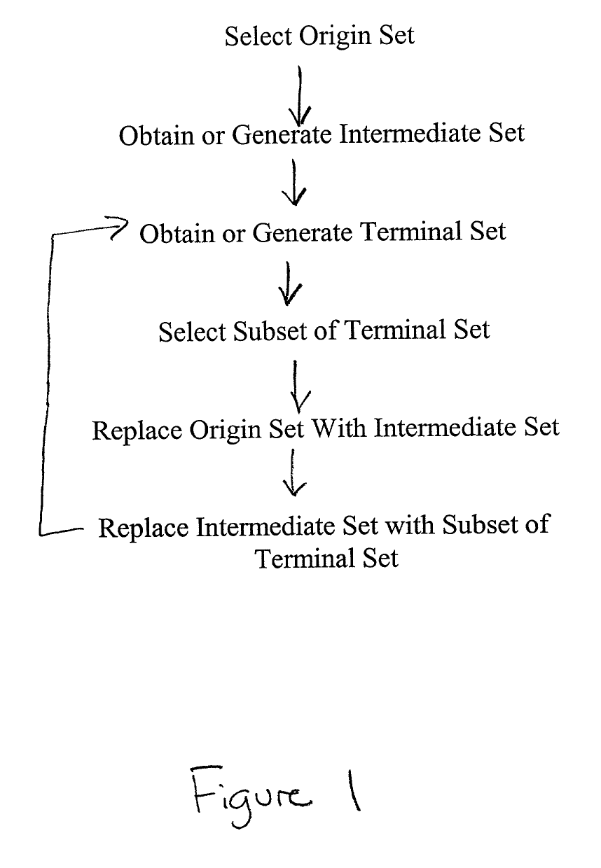

FIG. 1 shows an example of a general process for generating or obtaining members of a targetshape group. Initially, an origin set comprised of a targetshape molecule is selected. In the next step, an intermediate set of molecules is generated or obtained. In general, molecules of the intermediate set of molecules bind molecules belonging to the origin set. A terminal set of molecules is then generated or obtained. In general, molecules belonging to the terminal set bind to at least one molecule of the intermediate set. Next, a subset of the terminal set is selected. Generally, molecules belonging to the subset of the terminal set bind at least one member of the intermediate set by way of substantially the same epitope that the one member of the intermediate set binds at least one member of the origin set. In an iterative process, the origin set is replaced with the intermediate set and the intermediate set is replaced with the subset of the terminal set. Finally, the steps of obtaining or generating a terminal set, selecting a subset of the terminal set, replacing the origin set, and replacing the intermediate set can be repeated until a plurality of sets of molecules are obtained.

In general, members of any targetshape set si can be generated or obtained by screening a molecular diversity library composed of DNA, RNA, peptides, polypeptides, small molecules, or other compounds for molecules able to bind at least one member of a lower ordered targetshape subset si−1′. Alternatively, or in addition, members of si may be found by using members of targetshape set s(i−1)′ to raise antibodies against s(i−1)′. Random chemistry approaches beginning with molecules having a core structure similar to members of set s(i−2) may also be used generate candidate molecules for set s(i). Subsequently, members of a subset s1′ that compete for the same binding site on s(i−1)′ as members of s(i−2)′ may be discriminated from the remainder of set si. In this fashion, members of targetshape sets s0, s1, s2, s2′, si, si′, . . . , sn, sn′ maybe obtained.

The complete targetshape group of n sets of molecules forms a gradient in shape-function space surrounding the targetshape s0. The even rings i.e., sets of a targetshape group substantially correspond to shape mimics of s0 that are, on average, successively less like s0 as ring order increases, whereas members of a given odd ring substantially correspond to shape complements of molecules belonging to successive even rings. For example, because members of subset s4′ are identified by competitive binding with s2′ for sites on s3′, then, since s2′ members are similar to, but not identical to the targetshape s0, it follows that members of s4′ are generally more similar to members of s2′ than to s0. Consequently, the sets of molecules si, where i is even, comprise a gradient in the shape space surrounding s0 where members of a given subset si′ are less similar to s0 than members of the lower ordered subset s(i−2′).

By extension, the sets of molecules si, where i is odd, comprise a gradient in shape space surrounding s1 where members of set s(i+2)′ are less similar than members of lower ordered subsets si′ to the s1 shape complements of s0. It follows that molecules that bind members of odd numbered rings are successively more similar to s0, on average, as they bind to odd numbered rings of lower order. That is, for example, molecules binding members of s5′ are generally more similar to s0 than molecules binding members of s7′. Thus, the odd numbered sets provide a complementary shape “gradient” to select or screen for molecules ever more similar to s0.

In selecting members of a given subset si′, odd or even, it may be advantageous to proceed by competitive displacement of members of si off s(i−1)′ using only members of s(i−2)′. Alternatively, members of set si′ may be selected by competition with members of any lower ordered subset s(i−k)′ where i−k≧0 and k is even. Choosing k>2 results in a less-steep gradient because successive subsets are chosen by competition with molecules that are somewhat more similar to either s1 or s0. The value of k need not be the same for obtaining successive subsets. Thus, the choice of k allows the gradient in shape function space between successive sets of molecules to be tuned at each step in the process.

The steepness of the gradient surrounding s0 or s1 may also be modified by setting more or less stringent competition or binding requirements for entry into a given set or subset.

5.4.1 Multiple Target Problems

The ability to detect lead candidates that do not bind a target efficiently enough to identify by available screening techniques allows the method described above to be extended to address the multiple target problem. Each characteristic that the molecule must posses or criterion that the desired molecule must satisfy can be considered a “task.” Without loss of generality, and as an example only, consider the problem of finding a molecule able to accomplish two tasks such as having the ability to bind receptors of both estrogen and progesterone. Defining estrogen as s0, one may obtain a targetshape group of n sets molecules corresponding to targetshape rings around estrogen. Similarly, define progesterone as r0 and obtain sets of molecules corresponding to targetshape rings around r0. The targetshape groups surrounding each targetshape need not contain the same number of sets of molecules.

A molecular diversity library is then screened for molecules that bind at least one member of an odd ring of both targetshape s and targetshape r. Alternatively, or in addition, members of at least one even numbered ring of targetshape s and/or targetshape r may be used to raise antibodies against the even numbered ring. The antibodies are then screened for molecules that bind at least one odd ring of both targetshape s and targetshape r. Consider a molecule X that binds to s3′ and also to r5′. By way of example, suppose that X does not bind to s1 nor to r1. Thus, X would remain undetected using conventional screening tests for molecules binding only the equivalents of targetshape sets s1 and r1. Conversely, using the full targetshape groups surrounding estrogen and progesterone candidates that are only somewhat similar to both estrogen and progesterone may be found.

To obtain compounds with improved binding to both the estrogen and progesterone receptors, variants of X are obtained or generated, for example, using a molecular diversity approach or other random chemistry approach. Alternatively, or in addition, molecules in the same region of shape space as is X are generated or obtained. The new population of molecules is screened to identify members that are more similar to both estrogen and progesterone as ranked by the ability to bind members of lower ordered odd numbered rings of targetshape s and/or targetshape r. Thus, a molecule binding at least one member of s3′ and r5′ is an improvement of X, for it is more similar to progesterone and as similar to estrogen. A molecule able to bind at least one member of s1 and r3′ is better than X in being more similar both to estrogen and to progesterone.

The screening power of this extended targetshape approach is particularly advantageous in situations where one cannot sample molecular shape space with sufficient density to identify immediately candidates, if they exist, that bind both s1 and r1 . For example, although a molecular diversity library may contain approximately 10⁻¹⁵ distinct molecular species, the chance of identifying a potential candidate to mimic both s0 and r0 may be slim. However, using higher order sets of a targetshape group instead of one or a small number of target compounds, a broader region of molecular shape space can be sampled with high density to identify proto-candidates even only somewhat similar to both s0 and r0. Subsequently, those candidates can be used to generate a molecular diversity of shape variants focused around the same general region of shape space as the initial candidates. Thus, about 10¹⁵ molecules in that “region” of shape space can be created and screened or selected upon for improved variants. The improved variants can then be used to create still further variants, in an attempt to increase similarity to both s0 and r0.

The process described above can be generalized to seek molecules able to fulfill a plurality of arbitrary “Boolean” or logical combinations of “yes” and “no” conditions on different targets or different criteria. For example, one may seek a molecule that binds one hormone receptor but does not bind the receptor of another hormone, or one may seek a molecule that binds a cis acting promoter of one gene but does not bind the cis acting promoter of another gene, etc. In general, n targetshape groups, r, s, t, . . . , n would be constructed for each of n tasks. Candidates only partially fulfilling one or more of the tasks may be identified. Improved candidates could be sought by obtaining variants better able to fulfill each task separately, or by obtaining variants better able to fulfill any subset of the n tasks. Then, the initial candidates may be optimized to seek the practically accessible pareto optimal set.

For example, consider a multitask problem that includes obtaining molecules that bind at least one target, and do not bind at least one other target. As a specific example only, consider seeking a molecule that binds the estrogen receptor but does not bind the progesterone receptor. Targetshape groups are constructed around estrogen, s0, and progesterone, r0. Initial candidates are sought that bind to low ordered odd rings of targetshape s and either will not bind any odd ring of targetshape r, or will only bind higher ordered odd rings of targetshape r so that it is unlikely to interact with or bind the progesterone receptor. However, an initial candidate may bind at least one lower ordered odd rings of targetshape r. By successively generating variants of the initial candidates and selecting improved candidates that bind primarily to higher ordered odd rings of targetshape r, variants of initial candidates are “tuned” away from similarity to progesterone or interaction with the progesterone receptor. The ability of a variant to bind lower ordered odd rings of estrogen may also be considered when selecting improved variants to continue the optimization procedure. Thus, targetshape provides a means to “sculpt” molecules to enhance the ability to bind the receptor of one molecule while decreasing binding to the receptor of a second molecule. In this example, even subtle side effects due to undesirable binding of a drug to the progesterone receptor would be avoided.

The initial candidates X can be partially ordered based upon their binding to rings of one or more of targetshape groups. It may be advantageous to order fully the candidates according to a system that assigns different weight to the ability to accomplish each task. The candidates can also be ordered based on the absolute or relative number of members of a given targetshape ring that bind each candidate and/or the relative strength or efficiency of binding. However, it is not necessary, even to optimize the ability to accomplish multiple tasks to create a full ordering relationship between all candidate molecules.

A pareto optimal set of candidates, X, is defined as a set of molecules having the property that no other molecules exist that are better than the members of the pareto optimal set with respect to at least one “task” and at least as good with respect to the remaining tasks. In the case where only a partial ordering exists, the pareto optimal set constitutes the “end point” of the effort to find good candidates for both tasks. The pareto optimal set is defined for the set of all possible molecules. Thus, in reality, it is impossible to assure that a candidate pareto optimal set, X, is actually pareto optimal.

In the current context, a pareto optimal set of candidates for the tasks of being similar to estrogen and also to progesterone is a set X such that no other molecule exists that is more similar to estrogen, and at least as similar to progesterone; or that is more similar to progesterone and at least as similar to estrogen.

5.5 Directed Chemical Reactions

It is well established in the art that it is possible to generate novel enzymes, for example, catalytic antibodies, by obtaining antibodies that bind the transition state of a reaction, as disclosed in S. Pollack, J. Jacobs, and P. Schultz, Science, 1986, 234:1570, A. Tramontano, K. Janda, R. Lemer, 1986, Proc. Natl. Acad. Sci. USA, 83:6736, A. Tramontano, K. Janda, R. Lemer, 1986, Science, 234:1566, and U.S. Pat. No. 4,888,281 to Schochetman et. al, incorporated herein by reference in their entireties. For example, consider a specific reaction such as the conversion of a substrate A to a product B. Enzymes that bind the transition state, A*, are candidates to catalyze the conversion of A to B. Because A* is often unstable it cannot be used to raise antibodies that bind A* and, therefore, might serve as catalysts for the reaction of A to B. However, immunization with stable analogues of the transition state typically yields some monoclonal antibodies that are able to bind A* and hence do catalyze the conversion of A to B.

It is an aspect of the present invention that the rings of a targetshape group may be used to direct and catalyze chemical reactions to generate molecules having predetermined characteristics. The even rings of a targetshape group substantially correspond to shape mimics of s0 that are, on average, successively less like s0 as ring order increases, whereas members of a given odd ring substantially correspond to shape complements of molecules belonging to successive even rings. Thus, members of a given higher ordered odd ring of a targetshape group substantially correspond to shape complements of one or more molecules, Ai, that become successively less like s0 as the order of the ring increases. Given that members of the odd ring bind the molecules Ai, the members of the odd ring are candidate catalysts, e.g., enzymes, to catalyze conversion of the Ai, taken as substrates, to form one or more product molecules Bi. Because the product molecules Bi comprise reaction products of substrates Ai, a given product molecule Bk will likely exhibit similarities to and differences from its parent substrate molecule Ak. Consequently, Bk may either be less similar to s0 than substrate Ak or Bk may be more similar to s0 than Ak.

If Bk is more similar to s0 than Ak, then Bk is likely to correspond to a shape complement to at least one member of a lower ordered odd ring than Ak. Consequently, it is likely that at least some members of the lower ordered ring bind Bk so that the members of the lower ordered odd ring are candidates to catalyze the conversion of Bk, taken as a substrate, to form one or more further products Ci. In general, a given product molecule Ck, may be more or less similar to s0 than the parent substrate Bk. Thus, members from successive odd numbered rings are potential catalysts for a succession of reactions whereby product molecules increasingly similar to a given target molecule are created. Thus considered, the odd numbered rings comprise a kind of catalytic funnel converting a wide diversity of initial substrates into products directed towards shape features more or less similar to s0.

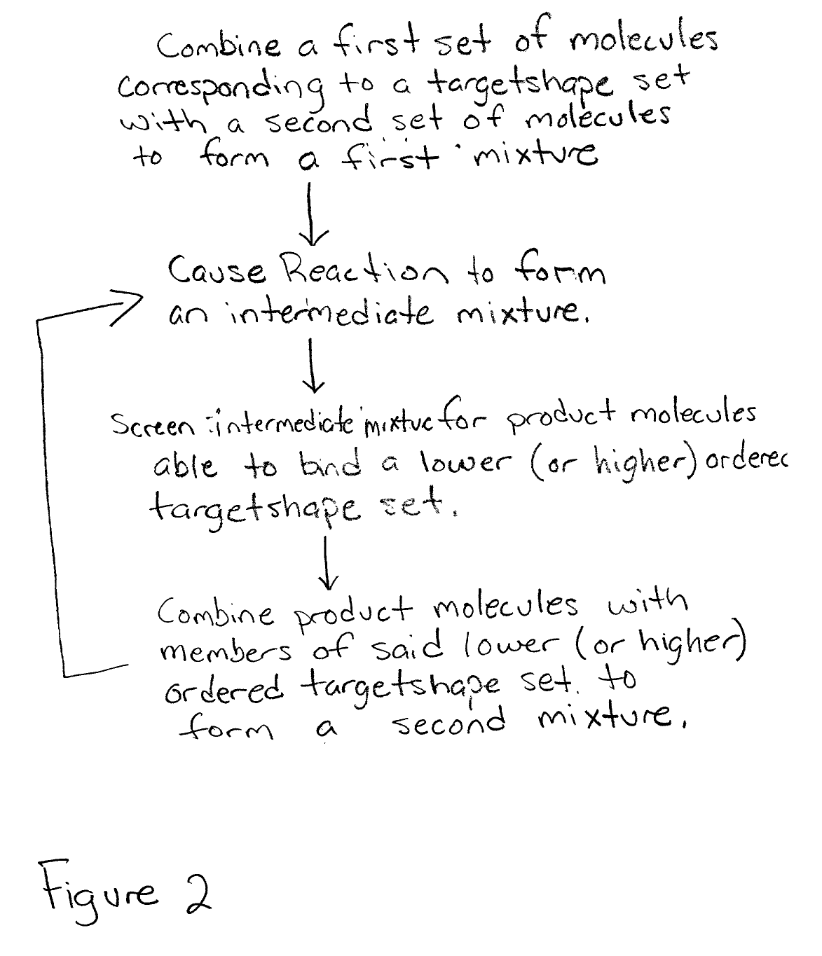

The directed chemical reactions require a set of potential substrates to be acted upon by members of the odd numbered rings. Higher ordered even numbered rings corresponding to the odd numbered rings comprise such a set of potential substrates. However, additional substrate candidates may be obtained by using the odd rings to screen any molecular diversity libraries such as DNA, RNA, peptide, or small molecule libraries to obtain substrates that bind to members of the odd rings. Substrate candidates may also be generated by using members of the odd numbered rings to raise antibodies against the odd numbered rings. Such a process, which is equivalent to identifying new members of the even numbered rings, s0, s2′, s4′, . . . sN′, identifies further candidate substrates upon which the odd numbered rings, the catalytic funnel, can act as catalysts to generate a diversity of molecules like S0. FIG. 2 is a flowchart representing an example of a process for generating a series of directed chemical reactions. Initially, molecules corresponding substantially to members of an odd targetshape set are combined with a plurality of molecules able to bind at least one member of the odd targetshape set to form a first mixture. A reaction is caused to take place in the first mixture to form an intermediate mixture having at least one molecule different from the first mixture. The intermediate mixture is screened for product molecules that bind a lower (or higher) ordered odd targetshape set. The product molecules are combined with molecules corresponding to members of said lower (or higher) ordered odd targetshape set to form a second mixture. The first mixture is replaced with the second mixture. The steps of screening, combining product molecules, and replacing may be repeated until a product molecule having a desired characteristic is identified.

Thus, one can screen for proto-candidates that bind to molecules that correspond to members of higher order sets of a targetshape group. From the set of proto-candidates, variants are created and tested in an iterative process to find improved candidates binding to molecules that correspond to members of successively lower ordered sets of the targetshape group.

As an example only, consider again the multitask problem of finding a molecule able to bind both the estrogen and progesterone receptor. Targetshape groups centered around estrogen and progesterone may be used to find a candidate molecule X that binds to odd numbered S and odd numbered R rings. Odd numbered rings of a targetshape group centered around X are candidates to catalyze a sequence of reactions to produce product molecules focused into the region of shape space occupied by X. The product molecules may be screened for improved candidates that bind lower ordered odd rings of targetshape r and targetshape s. The process may be repeated by constructing targetshape groups around the selected product molecules. These targetshape groups are also candidates to catalyze a sequence of reactions to produce product molecules focused in a region of space occupied by molecules that bind both estrogen and progesterone. Consequently, the present invention allows production of shape variants of any molecule that are focused into a predetermined region of shape space.

At any point in the process, it may be desirable to determine the structure or functional properties characterizing any of the candidate substrates, catalysts, or reaction products that are identified during the sequence of reaction and screening steps. It may also be desirable to generate a succession of new targetshape groups centered around improved candidates identified at any step during the process.

Existing random chemistry techniques (see, in particular Patent No. WO9424314 to S. Kauffinan and J. Rebek) can be extended by combination with the catalytic funnel of the odd numbered rings. Random chemistry consists in confronting a random library of substrates with a set of catalysts that acts on members of the substrate library to generate a new library of products. The new library is acted upon by either the same set of catalysts or a different (but possibly overlapping) set of catalysts to generate a further library of products. This process can be iterated, and screening or selection for useful products carried out at any point. One a product have desirable characteristics is identified, winnowing the substrates and catalytic sets allows identification of the substrates and enzymes that are essential to formation of the product.

Thus, the odd rings of a targetshape group can be used with a wide variety of other substrates and catalysts to generate candidate substrates of members of an odd ring of the targetshape group. Such a process is equivalent to generation of new members of higher ordered even rings of the targetshape group. Thus, random chemistry, directed by catalytic funnels, provides one means to generate numerous candidate substrates in a desired region of shape space.

Consequently, regarding a multiple task problem, the present invention provides a method for identifying an initial, perhaps poor, candidate able to satisfy at least two criteria and further provides a means to populate the specific region of shape space surrounding the candidate molecule with many molecules from which improved candidates can be selected.

The process of directed chemical reactions may be extended to seeking molecules able to catalyze the reaction of at least two different substrates by seeking molecules able to bind the transition state A* of a first reaction and the transition state, X*, of a second reaction. Alternatively, targetshape can be used to seek molecules that catalyze one reaction and do not catalyze a second reaction. In general, molecules can be sought which satisfy a number of Boolean logic statements regarding criteria related to catalysis, inhibition, binding, localization, transportation, and other tasks.

5.6 Characterization of Molecular Shape Space

The concept of shape space has been discussed in Perelson and Oster, 1979, J. Theor. Biol. 173:649 (Perelson), and reviewed in Stuart Kauffman, The Origins of Order, Oxford University Press, New York, 1993, references which are hereby incorporated in there entirety. Perelson showed that features of molecular epitopes could be represented in an N-dimensional shape space where the N dimensions correspond to spatial coordinates defining molecular geomtery and physio-chemical features such as charge, dipole moment, hydrophobicity etc. Each feature of a given molecule is assigned a value along one of the N axes. For realistic molecules, each feature has a minimum and a maximum value, hence shape space is bounded and compact. The dimensionality of shape space may be between about three and about ten and is, preferably, about 5 dimensional.

The present invention provides a method for coordinating the relative positions of molecules in within such an N-dimensional shape space where the N dimensions correspond to spatial coordinates defining the molecular geometry and physio-chemical features such as charge, dipole moment, hydrophobicity etc.

Random peptides from phage display used to screen for molecules that bind arbitrary antigens indicate that about one in a million random peptides will bind any given antigen. Consequently, a library of about 1,000,000 random peptides would suffice to find at least one peptide that binds any arbitrary molecule, e.g., antigen. Thus, molecular shape space can be described using about 1,000,000 distinct volume elements.

As described above, each targetshape group is comprised of successive sets of molecules corresponding to increasingly similar shape complements or mimics of the central molecule as ring order decreases. For example, consider targetshape groups constructed around two at least somewhat dissimilar molecules. It is unlikely that molecules corresponding to a low ordered even or odd ring from one targetshape group would bind molecules corresponding to molecules of a low ordered odd or even group of the other targetshape group. However, it is more likely that molecules corresponding to higher ordered even or odd rings from one targetshape group will bind molecules that correspond to members of higher ordered odd or even rings from the other targetshape group. Targetshape groups satisfying this condition are said to intersect or interpenetrate. If a molecule binds members of at least one ring of two targetshape groups, the position of that molecule relative to the targetshapes may be determined. Constructing enough intersecting targetshape groups provides a means of establishing the relative position in shape space of any arbitrary molecule.

For example, consider a 5-dimensional shape space and assume that one million effectively different molecular shapes are sufficient to bind to any molecule. Such a shape space may be completely described using 5-dimensions each having 12 resolution elements or about 5¹² total resolvable elements. Consider constructing targetshape groups around two distinct targetshapes each having characteristics at elements 4 and 8 along each axis. By definition, molecules corresponding to higher order even rings of each targetshape group will lie at positions along each axis that are different from molecules at the centers of the respective targetshape groups. Therefore, it is likely that some members of a higher ordered even set from one targetshape group will bind some members of an odd set from the other targetshape group. Thus, the relative position of any molecule which binds at least one even ring of one targetshape group and at least one odd ring of the other target shape group can be established.

If shape space is 5 dimensional, then about 2⁵, or 32 evenly positioned targetshape groups, each to about the 4^{th} order, should suffice to identify the relative position of any arbitrary molecule. Since the center molecules of the targetshape groups are randomly positioned in shape space so that lower ordered rings of some targetshape would overlap, about 100 targetshape groups would suffice to coordinate almost all of shape space. Each targetshape group, prepared using current molecular diversity technology would require about 9 man months to complete experimentally. If so, then between 100 and 300 man months of work would suffice.

A more refined analysis of relative positions within and between targetshape groups can he achieved based the relative affinities, e.g., binding affinities of a given molecule for members of a single or adjacent targetshape groups. For example, consider a family of receptors and ligands. The relative affinities of a given ligand for each receptor can be viewed as a measure of distances between the ligands. Reciprocally, the affinities of the set of receptors for any ligand can be used to construct a distance measure among any pair of receptors. In short, using these affinities, it is possible to more precisely “coordinate” the relative locations of the receptors or ligands with respect to one another, or the relative locations of the ligands with respect to one another.

Thus, the even and odd sets of nearby targetshape groups comprise just such sets of receptors and ligands. Thus, within and between the rings of different targetshape groups, more precise measures of the relative locations of any given molecule in shape space can be obtained.

A coordinated shape space is of high value, for example, in drug discovery. For we can now take any small molecule, see to which molecules it binds, and then construct a family of similar small molecules and test for preferred directions of movement in shape space to seek molecules that bind a preferred target.

5.6.1 Treatment of Autoimmune Disease

Autoimmune diseases arise because self antigens induce an antibody response. Current best knowledge states that immune memory is carried by immune system memory cells, which are formed in a process defined as “maturation of the immune response”. Characteristically, a novel antigen enters the host. Cells called B cells that produce and display antibodies that bind to the antigen with high affinity are thereby stimulated to proliferate faster. A hypermutation mechanism sets in and mutates the rearranged antibody genes, V, T, J, D, H, thereby mutating both the several (5 or 6) complement determining regions in the antibody molecule, where each CDR is about 8 to 15 amino acids in length, and mutating the surrounding “framework” amino acid sequences in the antibody molecule. Those B cells with mutated CDR and framework regions having a still higher affinity for the antigen are triggered to still more rapid proliferation.

In short, maturation of the immune response, typically happening over three weeks to several months depending upon exposures to the antigen, results in clonal selection of those founder B cells and their progeny whose antibody products have progressively higher affinity for the antigen. Typically, affinity increases several orders of magnitude, while the antibody gene accumulates an average of about 8 nucleotide substitutions, with a range from perhaps 2 to perhaps 25. The hypermutation mechanism shuts down after maturation.

Memory B cells are formed from matured B cells in an unclear manner. Current best knowledge states that memory cells, like naive B cells, can be induced to reinitiate the hypermutation mechanism. In particular, memory cells can be stimulated to hypermutate if exposed to an antigen modestly different, but not too different, from the initial antigen.

These facts underlie the invention which uses targetshape or other means to supply successively sets of “decoy” molecules having shapes sufficiently different from the initial antigen to induce the autoantibody memory cell population underlying an autoimmune disease to hypermutate and mature simultaneously towards the decoy antigen and away from the self antigen. Decoy molecules can be derived from a targetshape group or groups generated centered around either 1) the self antibody or antibodies or 2) the self antigen or antigens.

For example, consider a targetshape group centered around a0, a self antigen isolated from an animal having an autoimmune disease or response. Members of targetshape sets a2 and a4 differ from a0 in at least several different directions in shape space. Given a distribution of binding affinities of members of a targetshape ring for members of a corresponding target shape ring, the relative positions in shape space of the members of the targetshape group may be derived. Thus, vectors may be established that lead away in shape space from the self antigen a0 toward certain members of a2 and still further outward toward members of a4. Specifically, the relative metric would indicate the proper members of A2 as lying “between” A0 and a given A4 member. Thus, in general, with or without bothering to define absolute directions in shape space, we can “immunize” an autoimmune patient first with members of a2 followed by immunization with a4, or simultaneously with members of a2 and a4, or a2 alone, or members of a2, a4, a6, and outward toward higher order rings. The aim is to decoy the memory B cell population and their progeny away from the self antigen(s) a0.

Once the memory B cell population is decoyed far enough from a0 by use of members of the targetshape rings a2, a4, a6, . . . , an, A0 will no longer be able by itself to trigger return of the new memory B cells now centered on sites at A4 or A6, out of “range” for the hypermutation mechanism to find by maturation.

By extension, if the self antigen, a0, is not known, but the self antibodies, a1 are known, then they can be used to find the complementary ring, a2, thence a3 and a4. If the self antigen, a0, is unknown, it is possible that it is among the a2 set and cannot be discriminated. However, by construction, a0 is not among the a4 set. Hence, a4 can be used to decoy the memory B cell against the (unknown) a0. With some concern, one or more specific members of a2 can be used also to decoy the anti-aA0 memory cells, since almost all members of a2 will, in fact, differ from the true a0.

Where the memory B cell and its antibody is known, the therapeutic response to treatment with successive even or odd sets of a targetshape group can be followed by sequencing the corresponding antibody gene from clonal members, as is known in the art.

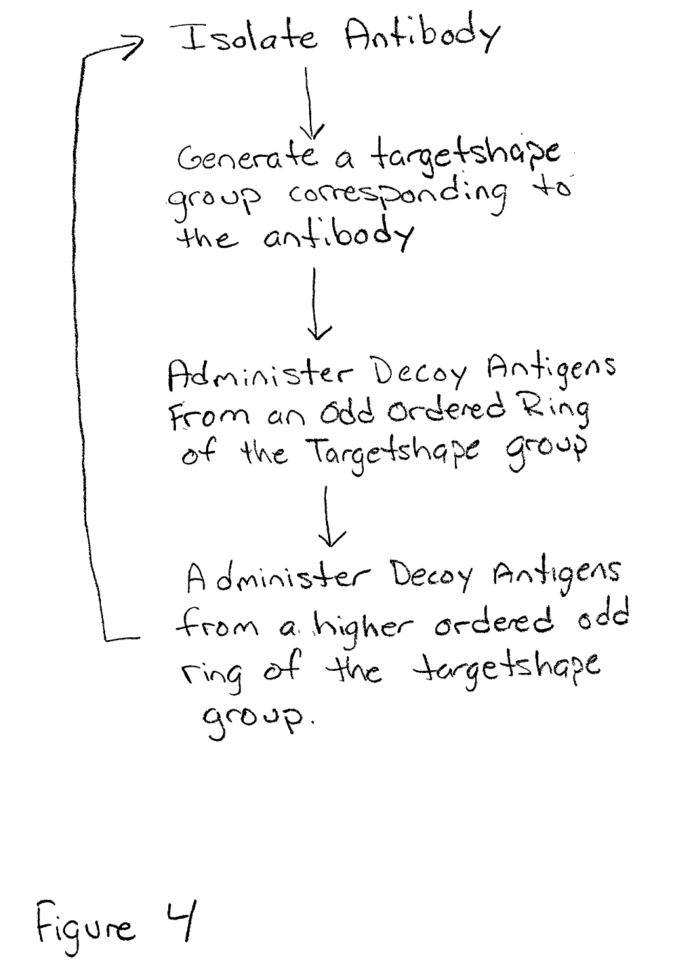

FIG. 4 is a flowchart representing a non-limiting example of a process for treating autoimmune disease beginning with an antibody derived from an animal having an autoimmune disease. One of ordinary skill in the art is aware that similar processes may be described beginning, for example, the self antibody or antibodies or the self antigen or antigens. The process may begin by isolating an antibody from an animal having an autoimmune disease. A targetshape group corresponding to the antibody is created using the present invention. Molecules corresponding substantially to members of a targetshape set si where i is odd, i.e., decoy antigens, are administered to the animal. Subsequently, molecules corresponding to higher ordered odd sets are administered to the animal. At any step in the process, as the antibodies of the immune system of the animal evolve, it may be desirable to isolate evolved antibodies from the animal, generate a second targetshape group corresponding to the evolved antibodies and repeat the steps of administering decoy antigens to the animal. Additionally, it may be desirable to determine the relative binding affinity between molecules of consecutive sets of the targetshape group so that the molecules belonging to each set may be ordered with respect to a relative distance in shape function space from the antibody. Treatment could then proceed by administering decoy antigens substantially in order of relative distance in shape function space from the antibody.

Thus, an embodiment of the present invention provides a method for treating an autoimmune disease comprising administering sets of decoy antigens which induce an autoantibody memory cell population underlying the autoimmune disease to hypermutate and mature towards the decoy antigens, wherein the sets of decoy antigens correspond substantially to members of at least one targetshape group.

5.7 Autonomous Agents

A molecular autonomous agent is a self reproducing physical system able to carry out at least one thermodynamic work cycle. All free living cells, e.g., bacteria, yeast, and cells in higher plants and animals, are autonomous agents in this sense.

An autonomous agent is, therefore, a non-equilibrium system. Recently, examples of reproducing molecular systems have been achieved by P. Bachman, et al, Nature, 1992, 357:57, Hartgerink, et al, J. Amer. Chem. Soc., 1996, 118:43, and Bag, et al, Pure Appl. Chem., 1996, 68:2145, which are hereby incorporated by reference in their entireties. These examples represent exergonic reaction systems that do not accomplish work.

Autonomous Agents in general, whether made of self reproducing light patterns in a laser, or of peptides and small organic molecules, are the subject of this invention. Since the biosphere is comprised of and has been built up by the co-evolution of such agents, their usefulness is enormous.

As a non-limiting example of utility, consider a molecular autonomous agent able to reproduce and evolve to catalyze a sequence of reactions leading to a desired small molecule or polymer with a desired property, for example, binding to the estrogen receptor, hence able to act as a drug lead, e.g., to mimic or modify the effects of estrogen.

Consider, for example, a small molecule, say estrogen, called s0. Use phage display, random chemistry, antibody generation or other procedures known in the art to find a set of, say peptides, all of which bind s0. Call this set s1. S1 are molecules that are the shape complement of s0. Now use s1 to bind a set of, say, peptides able to bind s1, called s2. The members of s2 may bind s1 at the same epitope that s0 binds s1, or at other epitopes. Choose only the former, e.g., by competition with s0 to show that s0 and s2 members compete for the binding site on a member of s1. Use this set of s2 to find a set s3, and repeat the process to find a set of consecutive targetshape sets. The even sets, s0, s2, s4, s6, . . . are all rather like s0 in shape, but become less like s0 as the set order increases from 2 to 4, to 6, etc. Conversely, the sets s1, s3, s4, s5 are the shape complements of the corresponding even set. Thus, the odd number set allows us to find molecules that are similar to s0 in shape, but not yet enough similar to s0 to bind s1. Thus, one may find a molecule, X, that binds at least one molecule in set S5. Then “mutant” or shape variants of X can be generated that may include molecules that bind s3, say a molecule Y. Shape variants of Y can be generated that may bind s1 and, hence, constitute sought for analogues of s0.

As noted above, the odd numbered sets or subsets, s1, s3, s5 also constitute a groups of complementary surfaces to the even numbered sets or subsets, s0, s2, s4. Consequently, the odd numbered sets constitute a set of candidate enzymes which can direct or focus a set of chemical reactions, for example as defined in random chemistry, or created in any other way, such that one or more substrate molecules undergoes reactions that include flow from shapes of small molecule substrates and products (or polymer substrate and products) similar to the even numbered shells inward, from s6 to s4 to s2 to s0. In short, odd numbered shells are a kind of catalytic “funnel” to take substrates and cause a web of catalyzed reactions centered on s0.

Now add the following: The complementary sets or subsets of odd and even numbered sets or subsets of peptides, s0, s2, s4 . . . and s1, s3, s5, . . . . are the shape complements of one another. Therefore the group of sets as a whole or many different subsets of such a group may well be capable of autocatalytic reproduction.

Consider any such autocatalytic set of, say, polymers with members from s1 to, say s6. This set is a set of reproducing complementary surfaces in which the odd ring polymers are also catalytic surfaces to catalyze the target shape like web of reactions centered on s0.

Consider any method to detect the presence of s0, say by binding to the estrogen receptor and changing the fluorescence of the estrogen receptor on some detector. We wish to use any such means of detection to supply a “selection” pressure to evolve and select for autocatalytic sets that create s0 like molecules, say small molecules, that bind to the estrogen receptor.

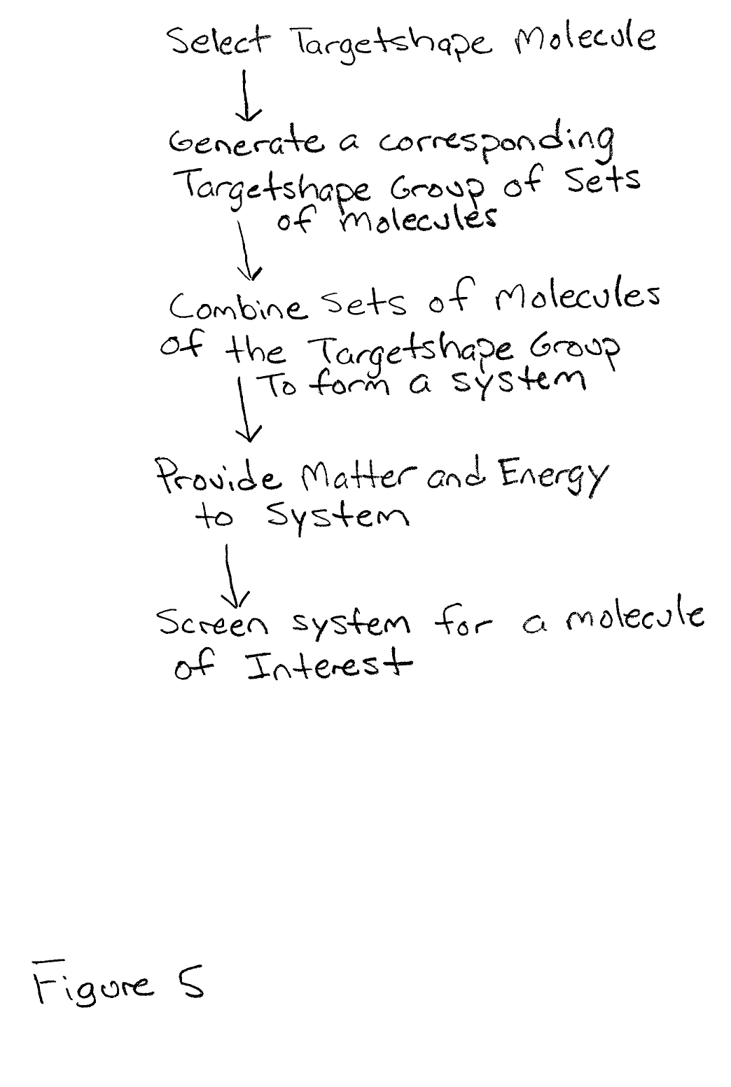

FIG. 5 shows a flowchart representing a non-limiting example of generating an autocatalytic system capable of producing a molecule of interest. The process begins by selecting a targetshape molecule. In general, the desired output of the system is a molecule having properties substantially similar or substantially dissimilar to the targetshape molecule. For example, if one wishes to generate molecules capable of binding the estrogen receptor, then estrogen might serve as the targetshape molecule. In the second step, a targetshape group of sets or rings of molecules corresponding to the targetshape molecule is generated using methods of the invention. The sets of molecules belonging to the targetshape group are combined to form a system. Next, the system is provided with matter and energy. Matter may be provided in the form of activated monomers, small monomers or any other molecule that may be acted upon by the system. Additionally, the system is provided with solvent or other media to allow or cause reactions to take place within the system. At some point, the system may be screened for a molecule of interest.

In general, our autocatalytic systems are open, non-equilibrium physical systems. Reproduction requires the addition of matter and energy, here called “food”, typically in the form of activated monomers or small polymers, or in any other way, such as photon flux, thermal energy, or electrical energy. We can, in a variety of ways, arrange for different reproducing molecular systems to compete, or be differentially rewarded with food. Without loss of generality, we might have our autocatalytic systems arrayed on a two-dimensional surface, detect the presence of s0 analogues as noted above, and differentially supply food to the regions on the two-dimensional array where autocalytic systems were producing s0 analogues.

A variety of approaches are known in the art for selectively positioning molecules within a two or three dimensional region. For example, U.S. Pat. No. 5,837,832, assigned to the Affymetrix Corp. and hereby included by reference in its entirety, describes one approach for producing two dimensional arrays of molecules on surfaces. Arrays of many thousands of oligonucleotide probes are synthesized on a substrate, such as a glass slide or chip. The method can be used, for instance, to synthesize “combinatorial” arrays consisting of, for example, all possible octanucleotides.

Whitesides et al Analytical Chemistry, 1998, 2280 have described microfabricating a plurality of wells in various substrate materials. The wells may be filled with liquids using discontinuous dewetting. Thus, such wells might serve as microfabricated reaction vessels in which food can be added in a selective fashion.

A method and apparatus for producing combinatorial position-addressable libraries of different-sequence oligomers or different-substituent small molecule compounds are disclosed by Dehlinger et al. U.S. Pat. No. 5,763,263, incorporated herein by reference in its entirety. The method employs massive parallel synthesis by stepwise subunit addition or substituent addition in a dense capillary-tube array. The libraries allow high throughput screening of library compounds in either solid phase or solution phase, and position-related identification of active library compounds. Similar methods position addressable methods can also be applied to planar substrates.

The differential reproduction of such systems, like the overgrowth of a bacterial colony with appropriate subplating or other techniques known in the art, will select out those self reproducing molecular systems that best produce s0 analogues.

Autonomous agents carry out work cycles. For molecular chemical systems, this means that exergonic and endergonic reactions are coupled, leading to the excess synthesis of the products of the endergonic reactions. Therefore, molecular autonomous agents in which the synthesis of s0 analogues is endergonic will have the property that those agents able to make s0 analogues via coupled exergonic and endergonic reactions, will produce an excess, even in large abundance of product—just as a red cell has a lot of hemoglobin.

The above description characterizes an invention capable of synthesizing a desired analogue of s0. This is of use to find, to identify, to characterize, and to produce such an analogue of s0.

A further feature of molecular autonomous agents, whether based on DNA, RNA and proteins as in normal prokaryotic and eukaryotic cells, or more generally, as a system of autonomous agents, is that such agents can evolve and co-evolve. Thus, we can select for improved variants with respect to synthesis of s0 analogues by means known in the art.

The present invention is not to be limited in scope by the specific embodiments described herein, which are intended as single illustrations of individual aspects of the invention, and functionally equivalent methods and components are within the scope of the invention. Indeed, various modifications of the invention, in addition to those shown and described herein will become apparent to those skilled in the art from the foregoing description and accompanying drawings. Such modifications are intended to fall within the scope of the appended claims. Various references including patent applications, patents, and other publications, are cited herein, the disclosures of which are incorporated by reference in their entireties.

What is claimed is:

1. A method for obtaining molecular diversity comprising the steps of:
obtaining an intermediate set of molecules that bind at least one molecule belonging to an origin set of molecules; and
obtaining a terminal set of molecules that bind at least one molecule belonging to the intermediate set of molecules.

2. The method according to claim 1 comprising the further steps of:
replacing the intermediate set of molecules with the terminal set of molecules; and
obtaining a new terminal set of molecules that bind to at least one molecule in the intermediate set of molecules.

3. The method according to claim 2 comprising the further steps of:
replacing the terminal set of molecules with the new terminal set of molecules; and
repeating the replacing the intermediate set of molecules step and the obtaining a new terminal set of molecules step.

4. The method according to claim 1 comprising the further steps of:
selecting a subset of the terminal set of molecules having a predetermined characteristic;
replacing the origin set of molecules with the intermediate set of molecules;
replacing the intermediate set of molecules with the subset of the terminal set of molecules; and
obtaining a new terminal set of molecules that bind to at least one molecule in the intermediate set of molecules.

5. The method according to claim 4 comprising the further steps of:
replacing the terminal set of molecules with the new terminal set of molecules; and
repeating the replacing the origin set of molecules step, the replacing the intermediate set of molecules step and the obtaining a new terminal set of molecules step.

6. The method according to claim 4 wherein the predetermined characteristic is the ability of a member of the terminal set of molecules to bind to at least one member of the intermediate set of molecules in substantially the same way as the member of the intermediate set of molecules binds at least one member of the origin set of molecules.

7. The method according to claim 4 wherein at least one binding assay is used to perform said selecting a subset of the terminal set of molecules step.

8. The method according to claim 1 wherein the obtaining an intermediate set of molecules that bind at least one molecule belonging to an origin set of molecules step comprises the step of:
screening a molecular library for molecules able to bind at least one member of the origin set of molecules.

9. The method according to claim 8 wherein the molecular library comprises DNA.

10. The method according to claim 8 wherein the molecular library comprises RNA.

11. The method according to claim 8 wherein the molecular library comprises peptides.

12. The method according to claim 8 wherein the molecular library comprises polypeptides.

13. The method according to claim 8 wherein the molecular library comprises small molecules.

14. The method according to claim 1 wherein the obtaining an intermediate set of molecules that bind at least one molecule belonging to an origin set of molecules step comprises the step of:
raising antibodies against the members of the origin set.

15. A method for drug lead discovery comprising the steps of:
creating a terminal set of molecules from an origin set of molecules using the method for obtaining molecular diversity as in claim 1;
producing one or more first variants of the terminal set of molecules that are similar to members of the origin set of molecules; and
selecting one or more of the first variants having at least one desired characteristic.

16. A method according to claim 15 comprising the further steps of:
producing one or more second variants of the first variants that are similar to embers of the origin set of molecules; and
selecting one or more of the second variants having the at least one desired characteristic.

17. A method for drug lead discovery comprising the steps of:
creating a terminal set of molecules from an origin set of molecules using the method for obtaining molecular diversity as in claim 1; and
obtaining a set of candidate molecules that are similar to members of the origin set of molecules.

18. A method according to claim 15 wherein the first variants comprise at least one of a stochastic sequence of polynucleotides.

19. A method for generating a molecule having a desired characteristic comprising the steps of:
obtaining a set of molecules that bind at least one molecule belonging to an origin set of molecules;
repeating said obtaining step from said set of molecules to generate a sequence of sets of molecules wherein the molecules in each of said sets bind to the molecules in a preceding one of the sets in the sequence;
combining one of said sets of molecules with a first group of molecules to create a plurality of product molecules;
selecting another of said sets of molecules in said sequence;
identifying one or more of said product molecules that bind at least one molecule in said selected set of molecules; and
repeating said combining step with said identified product molecules, said selecting step and said identifying step to generate the molecule with the desired characteristic.

20. A method of characterizing a first molecule comprising the steps of:
selecting a plurality of origin set molecules;
creating an intermediate set of molecules and a terminal set of molecules from each of said origin set molecules using the method for obtaining molecular diversity as in claim 1;
identifying terminal sets of molecules having at least one molecule that binds said first molecule; and
identifying intermediate sets of molecules having at least one molecule that binds said first molecule.

21. A method for treating an animal having an autoimmune disease comprising the steps of:
isolating an antibody from said animal to form an origin set;
obtaining a first intermediate set of molecules that bind said origin set;
obtaining a first terminal set of molecules that bind said first intermediate set;
obtaining a second intermediate set of molecules that bind said first terminal set;
selecting therapeutic molecules in said first intermediate set and molecules in said second intermediate set; and
administering said therapeutic molecules to said animal.

22. A method for producing an autocatalytic system capable of producing a molecule having a desired property comprising:
selecting an origin set of molecules;
generating an intermediate set of molecules and a terminal set of molecules using the method for obtaining molecular diversity as in claim 1;
combining said intermediate set and said terminal set of molecules to form a system;
providing matter and/or energy to said system; and
screening said system for molecules having said desired property.
